(19) United States
(12) Patent Application Publication
(10) Pub. No.: US 2023/0250394 A1
(43) Pub. Date: Aug. 10, 2023

(54) GENERATION OF PERINATAL-LIKE MESENCHYMAL STEM CELLS FROM HUMAN INDUCED PLURIPOTENT STEM CELLS

(71) Applicants: Zhen MA, Syracuse, NY (US); Tackla Simon WINSTON, Liverpool, NY (US)

(72) Inventors: Zhen MA, Syracuse, NY (US); Tackla Simon WINSTON, Liverpool, NY (US)

(73) Assignee: SYRACUSE UNIVERSITY, SYRACUSE, NY (US)

(21) Appl. No.: 18/012,733

(22) PCT Filed: Jun. 22, 2021
(86) PCT No.: PCT/US2021/038328

§ 371 Date: Dec. 23, 2022

Related U.S. Application Data
(60) Provisional application No. 63/043,257, filed on Jun. 24, 2020.

(51) Int. Cl. C12N 5/0775

(52) U.S. Cl. CPC C12N 5/0662; C12N 2506/45; C12N 2501/155; C12N 2501/119; C12N 2501/727; C12N 2500/90

(57) ABSTRACT

Non-naturally occurring perinatal mesenchymal stem cells differentiated from human induced pluripotent stem cells. The differentiation medium includes an amount of SB431542, an amount of BMP-4, and an amount of PD173074 and is repeated over the course of six days period until perinatal mesenchymal stem-like cells are formed. The resulting non-naturally occurring perinatal mesenchymal stem cells exhibit the relevant characteristics of naturally occurring perinatal mesenchymal stem cells, such as the positive expression of the mesenchymal stem cell markers CD73, CD90, and CD105. The non-naturally occurring perinatal mesenchymal stem cells may be used to produce paracrine factors and similar compounds for therapeutic purposes.

### CROSS-REFERENCE TO RELATED APPLICATIONS

The present application claims priority to U.S. Provisional Application No. 63/043257, filed on Jun. 24, 2020.

### BACKGROUND OF THE INVENTION

### 1. Field of the Invention

The present invention relates to perinatal-like mesenchymal stem cells and, more specifically, to an approach for differentiating perinatal mesenchymal stem cells from human induced pluripotent stem cells.

### 2. Description of the Related Art

Currently, the only source of perinatal mesenchymal stem cells is harvesting from various parts of the placenta and umbilical cord during childbirth, which limits their availability for therapeutic purposes. Moreover, the total cell number that can be isolated from these primary perinatal tissues are very limited, and difficult for scale-up manufacturing for clinical treatment of large patient population. Accordingly, there is a need in the art for an approach that can produce perinatal-like mesenchymal cells in a scalable manner so that those perinatal-like mesenchymal cells can be used for producing paracrine factors and similar compounds for therapeutic purposes.

### BRIEF SUMMARY OF THE INVENTION

The present invention provides an approach for differentiating perinatal mesenchymal stem-like cells from human induced pluripotent stem cells (hiPSCs) using a defined trophoblast cell stage, thereby allowing for large-scale production of perinatal mesenchymal stem cells for various therapeutic purposes, including the treatment of cytokine storms related to viral infections such COVID-19 infection. Perinatal mesenchymal stem cells primed by inflammatory conditions can release paracrine factors (e.g. extracellular vesicles, soluble factors) that exert profound anti-inflammatory and immunomodulatory effects on almost all the cells of the innate and adaptive immune systems. These cell products play several simultaneous roles including limiting inflammation through releasing cytokines, aiding healing by secretion of growth factors, altering host immune responses via immuno-modulatory proteins, and enhancing responses from endogenous repair cells.

The approach of the present invention is able to differentiate lineage-specific perinatal-like mesenchymal stem cells from hiPSCs. All of the biological processing is performed under serum-free condition, thereby ensuring clinical grade and good manufacturing practice (GMP) quality of cell products. The present invention thus provides for production of perinatal-like mesenchymal stem cells that can be used for the extraction of extracellular vesicles as a therapeutic solution for cytokine storm and sepsis conditions, thus avoiding concerns of immunogenicity and tumorgenicity. The present invention also enables the collection of pro-survival soluble factors for tissue repair and regeneration. The present invention may be implemented in large scale manufacturing with less cell population heterogeneity and biological variations.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a method for differentiating lineage-specific perinatal-like mesenchymal stem cells (pMSCs) from human induced pluripotent stem cells (hiPSCs) according to the present invention;

FIG. 2 is a series of phase-contrast microscopy images of hiPSCs, differentiated trophoblast cells, and perinatal MSC-like cells;

FIG. 3A is a series of immunofluorescent microscopy images with a 50 μM scale bar showing that hiPSC-derived trophoblast cells according to the present invention express the morphology of natural mesenchymal stem cells;

FIG. 3B is a series of immunofluorescent microscopy images with a 50 μM scale bar show that hiPSC-derived pMSC-like cells according to the present invention express the morphology of natural mesenchymal stem cells;

FIG. 4 is a graph of gene expression data showing the high expression of trophoblast related genes on the hiPSC-derived trophoblast cells;

FIG. 5A is a graph of flow cytometry data showing that hiPSC-derived pMSCs showed positive expression of CD105;

FIG. 5B is a graph of flow cytometry data showing that hiPSC-derived pMSCs showed positive expression of CD73;

FIG. 5C is a graph of flow cytometry data showing that hiPSC-derived pMSCs showed positive expression of CD90;

FIG. 6 is a pair of images showing the characterization of intermediate trophoblast stem cells with immunostaining of KRT7 and CDX2 markers;

FIG. 7 is a pair of images showing the characterization of intermediate trophoblast stem cells with immunostaining of GATA3, TEAD4, EPCAM and CDX2 markers;

FIG. 8 is a series of images showing the characterization of perinatal tissue mesenchymal stem cells with immunostaining of CD44, CD105, CD90, CD73, CD166 and CD144;

FIG. 9A is a graph of the characterization of perinatal tissue mesenchymal stem cells using flow cytometry;

FIG. 9B is a graph of the characterization of perinatal tissue mesenchymal stem cells using flow cytometry;

FIG. 10 is a gene expression analysis based on RT-qPCR showing cell differentiation progression from hiPSCs to intermediate trophoblast stem cells and then to perinatal tissue mesenchymal stem cells from passage 1 to passage 7 of the present invention.

### DETAILED DESCRIPTION OF THE INVENTION

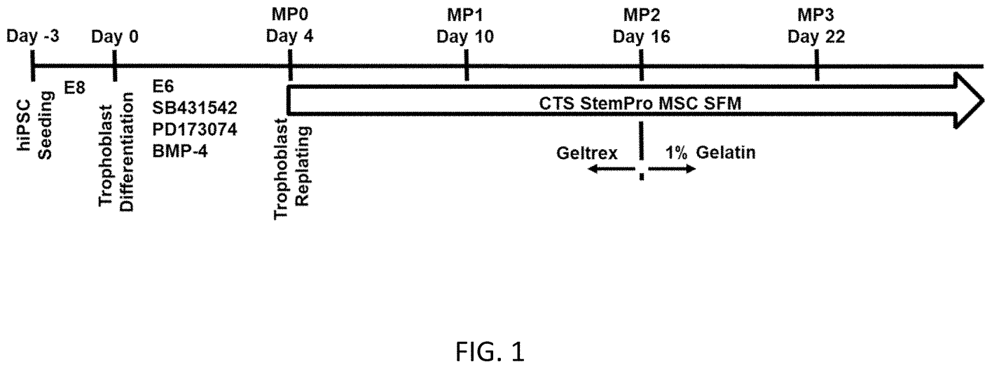

Referring to the figures, wherein like numeral refer to like parts throughout, there is seen in FIG. 1 a method for differentiating lineage-specific perinatal-like mesenchymal stem cells (pMSCs) from human induced pluripotent stem cells (hiPSCs) via the intermediate cell stage of trophoblast cells in a serum free culture media.

As an initial matter, the tissue culture surfaces must be prepared. As an example, a 6-well plate may be coated by adding a sufficient amount of a thin, gelled Geltrex solution to each well of the plates (about 1000 μL). Prior to seeding cells, the coated plates should be incubated for one hour at 37° C. The plates are removed after one hour and rested to allow the coating to equilibrate at room temperature for an additional 30 min to one hour. The plates may be stored by sealing with parafilm to prevent evaporation of the coating solution and refrigerated at 4° C. The plates can also be stored for up to one week.

As seen in FIG. 1, the method of the present invention begins with the step of hiPSC growth and expansion. hiPSCs are seeded and allowed to grow and expand for at least three passages in multiple wells within a 6-well plate using standard aseptic technique and pluripotent stem cell culture procedure. For seeding, cells that have expanded to 70-80% confluency should be used. Confluent hiPSC wells are washed with Dulbecco's phosphate-buffered saline (DPBS). The cells are then dissociated with 500-1000 μL Accutase® cell detachment solution per well for 4 min at 37° C. The Accutase® cell detachment solution is quenched with 3-4 mL of Essential 8 (E8) medium, prepared as described below, and the cell suspensions are transferred into a 15 mL conical tube. The cell suspension is then centrifuged at 54 g for two minutes, making sure the centrifuge is balanced. The supernatant is then aspirated and the cells are resuspended in 1 mL of the E8 media. 10 μL of the cell suspension is taken and added to a 1.5 mL microcentrifuge tube. To the tube, 10 μL Trypan Blue dye is added and mixed well (1:1 dilution). 10 μL of the cell/Trypan Blue mixture is loaded into a hemocytometer and the cells are counted accordingly. Once the cells have been counted, a cell suspension is prepared by diluting the initial suspension in E8 to bring the concentration of cells to a cell seeding density of 1.5×104 cells per cm². Sufficient Y-27632 rock inhibitor is then added to the cell suspension to bring the final concentration to 10 μM within the volume of cell suspension. Two mL of cells are then plated in each well. The plate is then agitated in an up-down, side-side fashion to evenly distribute the cells and incubated. This begins Day −2 of differentiation. The E8 media is changed to fresh E8 for the next day (Day −1).

As seen in FIG. 1, in a next step, the present invention comprises the step of trophoblast differentiation. After two days of hiPSC growth and expansion as described above (identified as Day 0 in FIG. 1), the E8 media is changed to use the E6 differentiation media prepared according to the present invention as described below. The E6 differentiation media comprises 10 μM SB431542, 10 ng/ml BMP-4 and 0.1 μM PD173074, and the preparation of each of these components is described below for reference. The E6 media is changed daily for four days.

As further seen in FIG. 1, the present invention next comprises the step of replating the trophoblasts on day four. To replate, the media is aspirated and the trophoblast wells are washed with DPBS. The wells are then dissociated with 500-1000 μL trypsin per well for 5-10 min at 37° C. The trypsin is quenched with 3-4 mL of the differentiation media described below and the cell suspensions are transferred into a 15 mL conical tube. The cell suspensions are then centrifuged at 500 g for five minutes, making sure the centrifuge is balanced. The supernatant is then aspirated and the cells are resuspended in 4 mL of a serum-free medium (SFM) specially formulated for the growth and expansion of human mesenchymal stem cells (MSCs), such as StemPro® MSC SFM CTS™ commercially available from Thermo-Fischer, referred to herein as the SF MSC medium. A cell suspension is then prepared by diluting the initial suspension in SF MSC to bring the concentration of cells to a 1:5 cell seeding density. Sufficient Y-27632 (rock inhibitor) is then added to the cell suspension to bring its final concentration 10 μM within the volume of cell suspension. 2 mL of cells are then plated in each well. The wells are agitated in an up-down, side-side fashion to evenly distribute the cells and then incubated. This begins passage number MP0 of differentiation.

As additionally seen in FIG. 1, several passages of differentiation are performed. For the next six days, the media is changed to fresh SF MSC media every two days. On day six, the cells begin passage MP1 by repeating the disassociation, re-plating, and incubation at a seeding density of 1:5. Another passage is performed every six days. Subsequent passages can be seeded at a cell seeding density of 2.0×104 cells per cm². In the event that cells do not detach during trypsinization, a cell scraper may be used. After a passage MP2, an attachment factor such as 1% gelatin can be substituted for Geltrex, and the rock inhibitor is no longer required.

As described above, the method of the present invention uses several different media, which may be prepared in advance. The media referred to as Essential 8 (E8) may prepared by aseptically adding the entire contents of E8 Supplement into a 500 mL bottle of E8 basal medium. The medium can be stored for up to 2 weeks in 4° C., or aliquoted into 40 mL working volumes and frozen in −20° C. for long term storage.

The 0.1 μM FGFR3 inhibitor (PD173074) solution may be prepared by diluting the contents of a source solution of FGFR3 inhibitor (PD173074) obtained from an off-the-shelf supplier in sterile dimethyl sulfoxide (DMSO) to make a stock solution at the desired concentration. The working volumes of small molecules are dependent on the volume of the E6 aliquots to dilute the PD173074 concentration to 10 μM.

The SB431542 ALK5 inhibitor solution may be prepared by diluting the contents of a source solution of SB431542 ALK5 inhibitor obtained from an off-the-shelf supplier in sterile DMSO. The volume of DMSO to add is specified by the supplier to make a 10 mM concentration. Working aliquots of the SB431542 solution can be calculated and stored at −20° C. The working volumes of small molecules are dependent on the volume of the E6 aliquots to dilute the SB431542 concentration to 10 μM.

The bone morphogenetic protein 4 (BMP-4) solution may be prepared by diluting the contents of a source solution of BMP-4 obtained from an off-the-shelf supplier in sterile 4 mM HCL containing 0.1% BSA to forms a 50-200 μg/mL stock concentration. Working volumes of the BMP-4 solution may be calculate and aliquoted, and then stored in −20° C. The working volumes is dependent on the volume of E6 aliquot to dilute the BMP-4 to a final concentration to 10 ng/mL.

The E6 differentiation media may be prepared by aliquoting the components of the E6 solution (10 μM SB431542, 10 ng/ml BMP-4 and 0.1 μM PD173074) described above into working volumes of 40 mL and then storing in −20° C. for long term storage. The differentiation media can then be thawed/stored in 4° C., but must be used within 2 weeks. When used, the media should be allowed to come to room temperature when in use (about 15-30 min after removal from refrigeration).

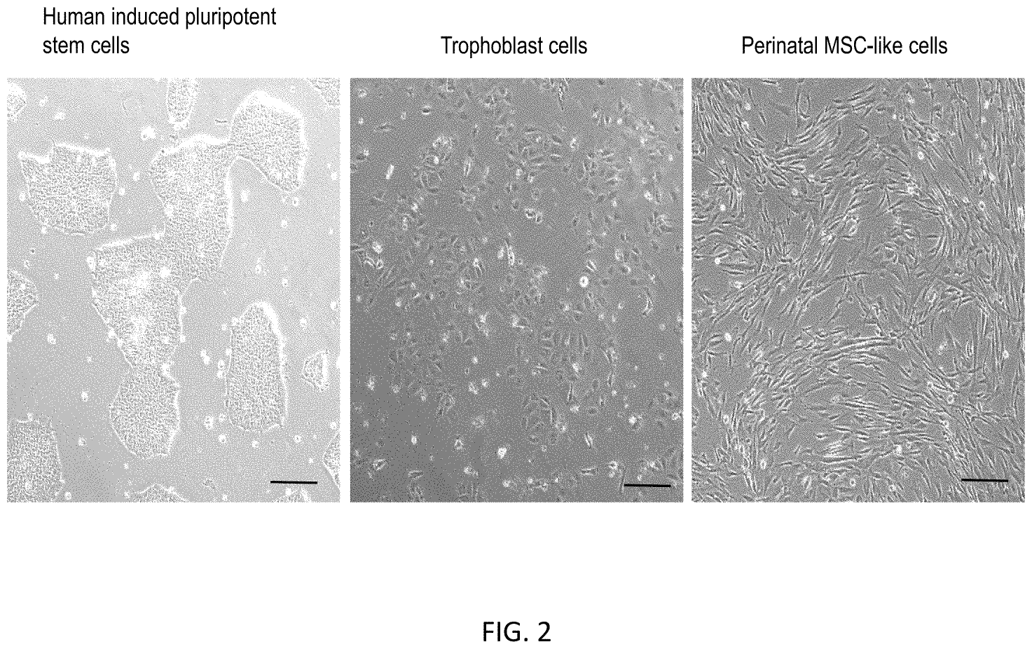

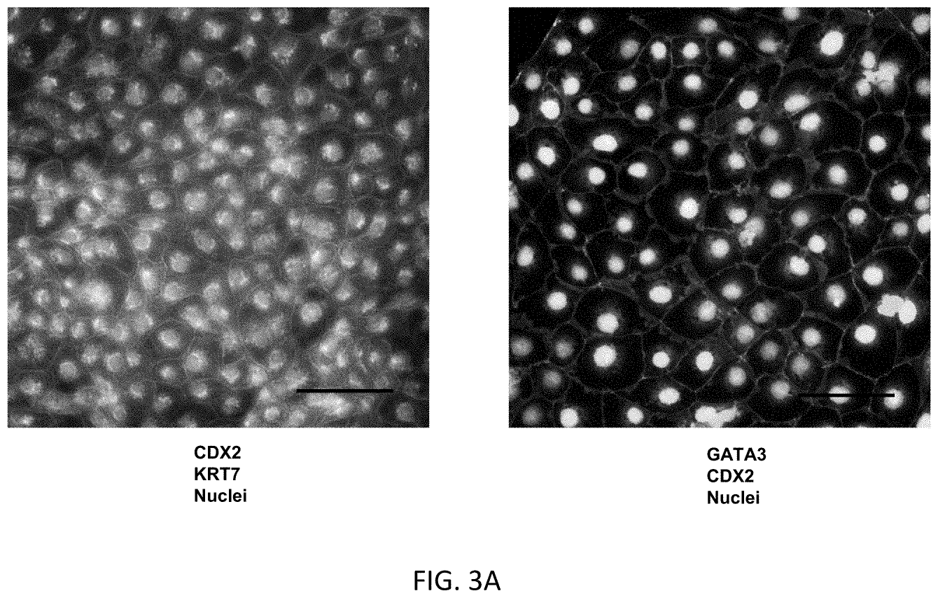

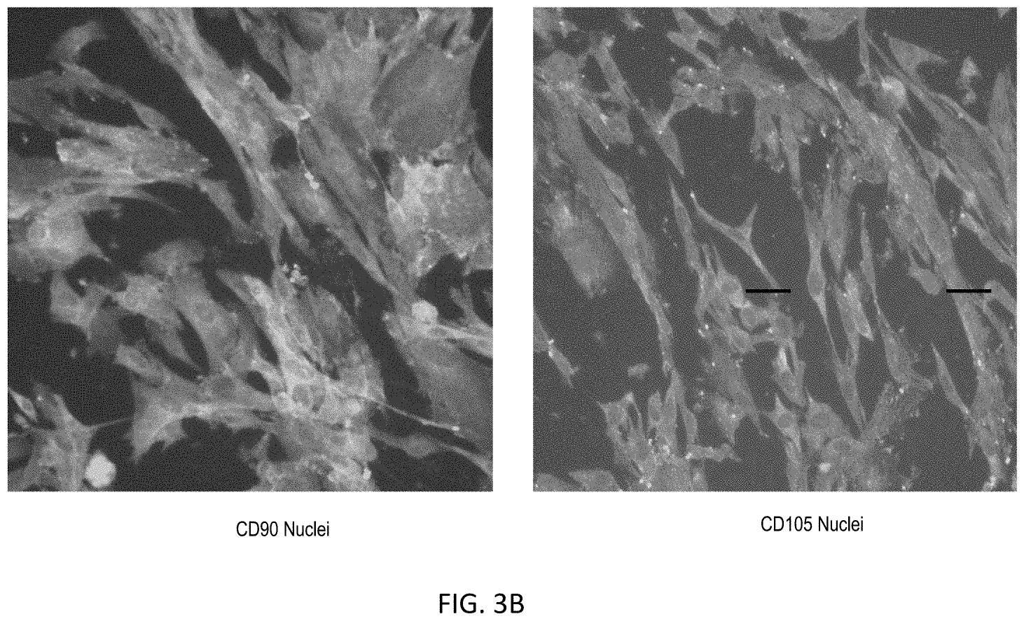

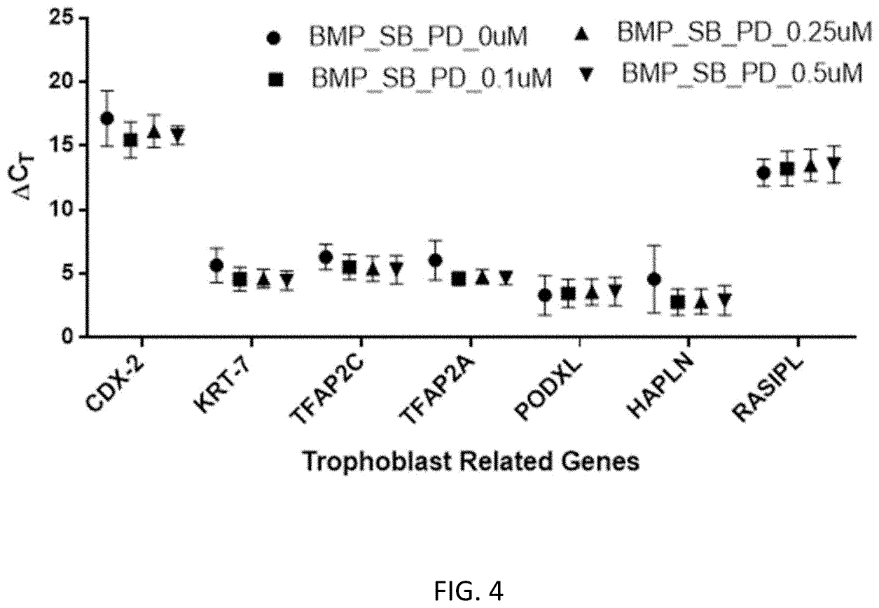

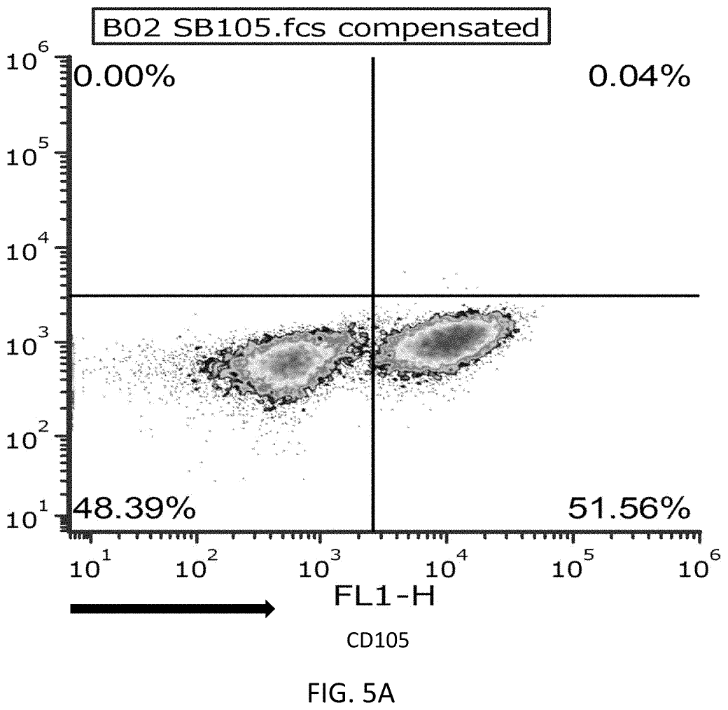

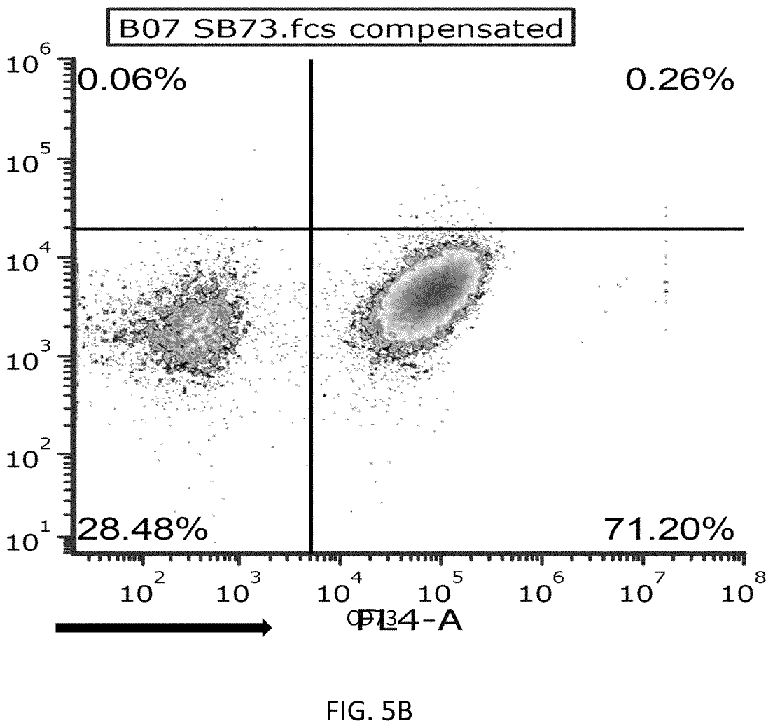

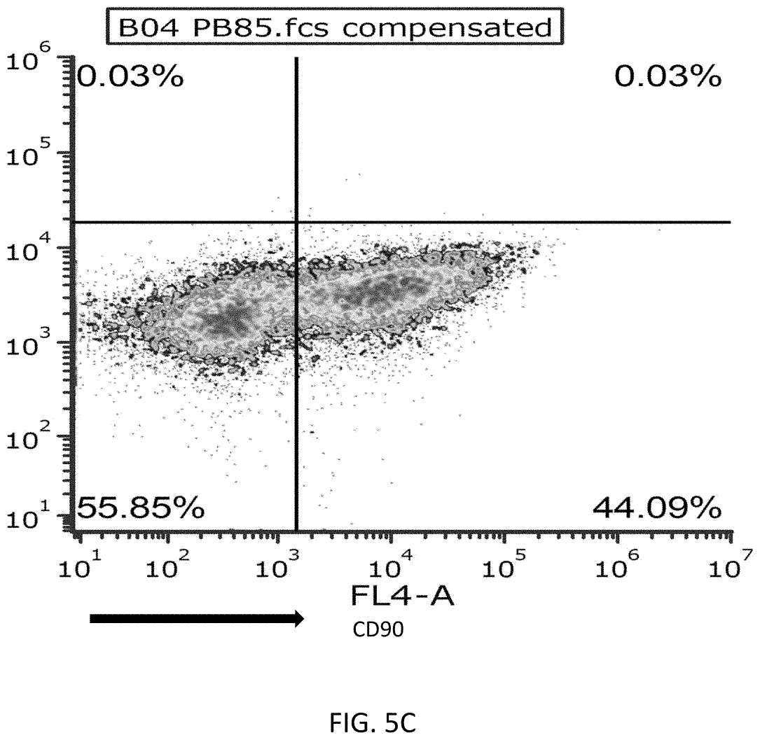

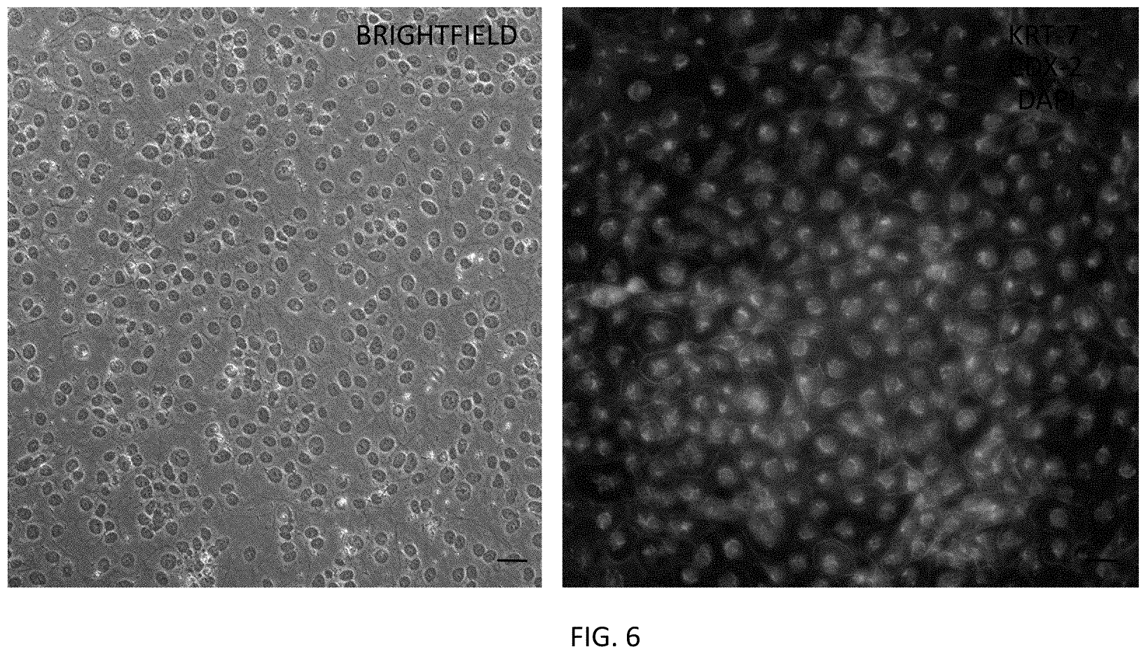

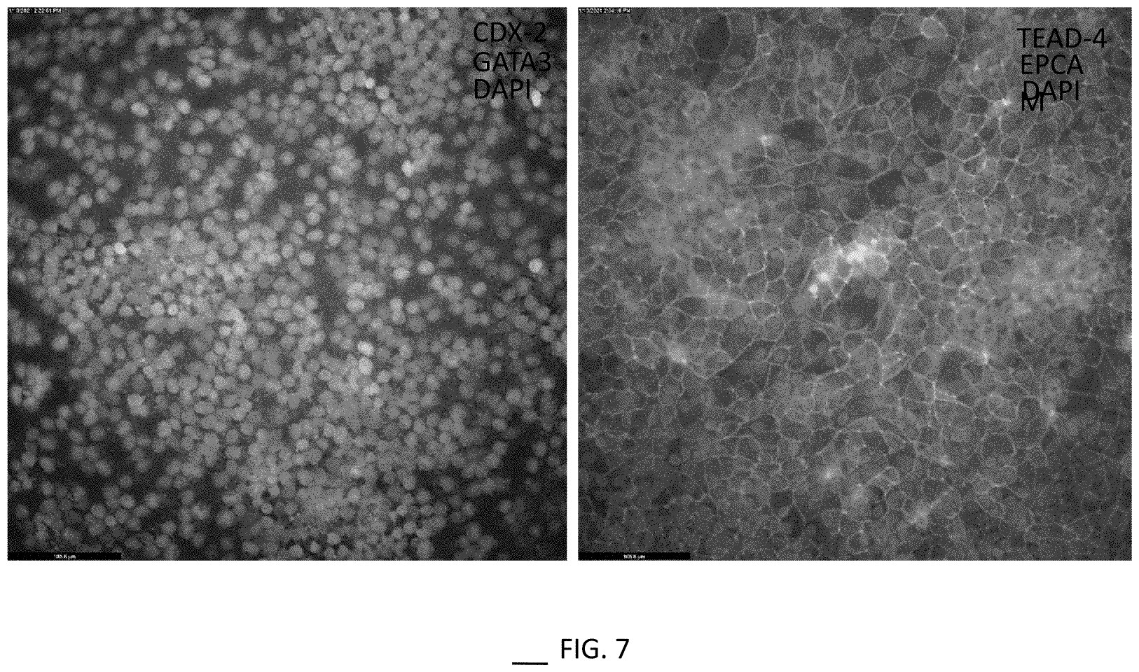

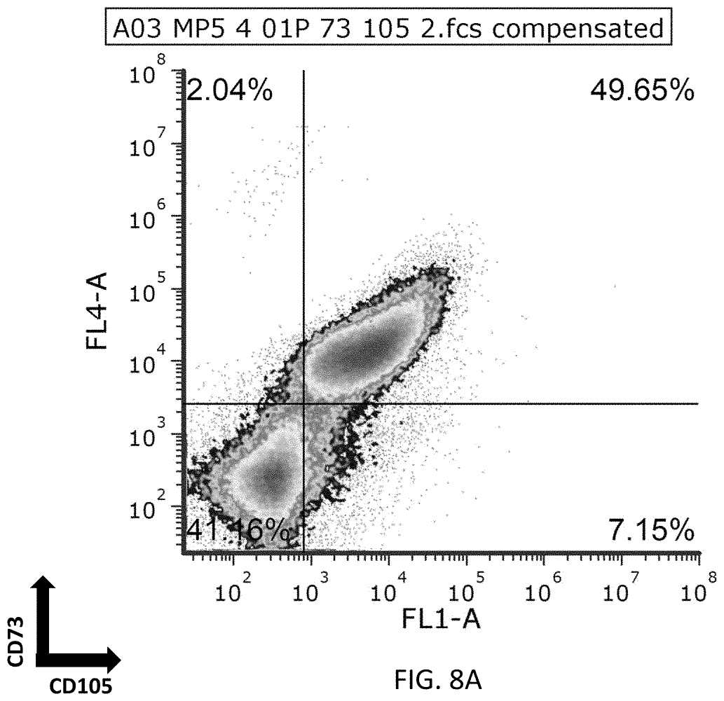

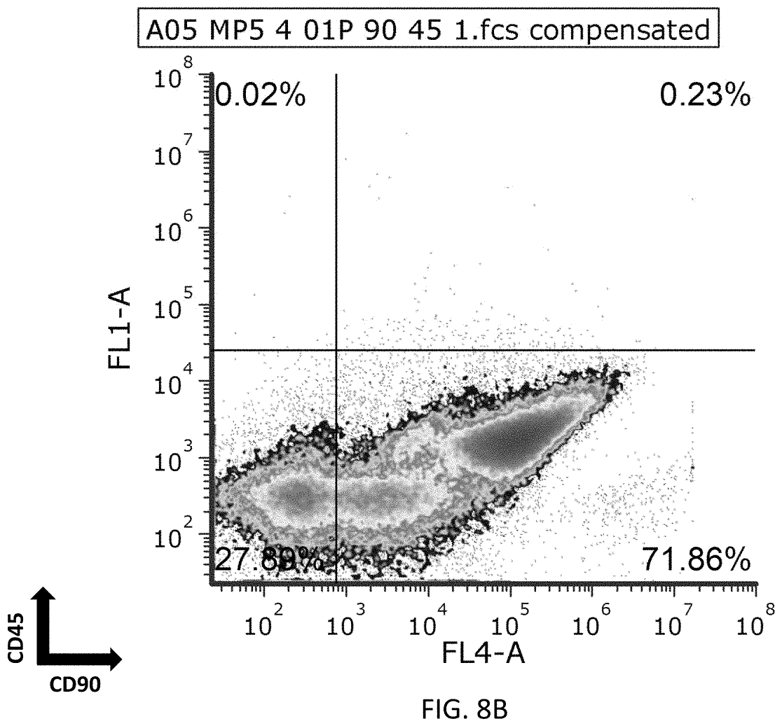

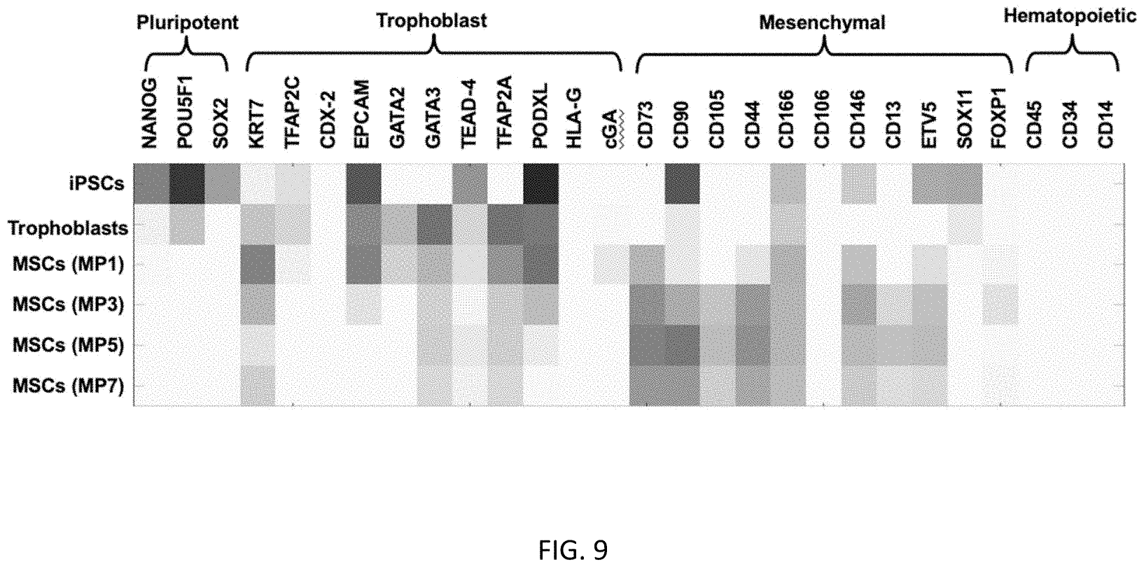

Referring to FIGS. 2 through 9, the non-naturally occurring perinatal mesenchymal stem cells differentiated from hiPSCs according to the method of the present invention exhibit the relevant and desired characteristics of naturally occurring perinatal mesenchymal stem cells. As seen in FIG. 2, microscopy images of hiPSCs, differentiated trophoblast cells, and perinatal MSC-like cells show a distinct cell morphology during the differentiation. The pMSC-like cells generated according to the present invention have spindle fibroblast-like morphology typical of actual pMSC cells. Referring to FIG. 3A, hiPSC-derived trophoblast cells expressed the typical trophoblast cell markers of CDX2, GATA3 and KRT7. As further seen in FIG. 3B, hiPSC-derived pMSC-like cells also expressed the typical mesenchymal stem cell markers of CD90 and CD105. As seen in FIG. 4, gene expression data showed high expression of trophoblast related genes on the hiPSC-derived trophoblast cells. As seen in FIG. 5A through 5C, flow cytometry data confirmed that hiPSC-derived pMSCs showed positive expression of CD73, CD90, and CD105, the generally accepted markers of mesenchymal stem cells. Referring to FIG. 6 and FIG. 7, further immunofluorescent characterization of intermediate trophoblast cells with immunostaining of KRT7 and CDX2 markers and GATA3, TEAD4, EPCAM and CDX2 markers, respectively, demonstrated the development of the desired activity. Referring to FIG. 8, characterization of perinatal tissue mesenchymal stem cells with immunostaining of CD44, CD105, CD90, CD73, CD166 and CD144 confirmed the approach of the present invention. As seen in FIGS. 9A and 9B, flow cytometry graphs characterizing perinatal tissue mesenchymal stem cells showed that differentiated cells from hiPSCs contain about 50% of cells that are double positive for CD73 and CD105. The differentiated cells contain about 70% of cells that are positive for CD90, and negative for CD45. Finally, as seen in FIG. 10, gene expression analysis based on RT-qPCR showed cell differentiation progression from hiPSCs to intermediate trophoblast stem cells, and then to perinatal tissue mesenchymal stem cells from passage 1 to passage 7. Perinatal tissue mesenchymal stem cells showed positive expression of mesenchymal markers, but negative for hematopoietic markers.

Perinatal-like mesenchymal stem cells (pMSCs) differentiated from human induced pluripotent stem cells according to the present invention can be used to prooduce high levels of anti-inflammatory secretome to help reverse the effects of septic conditions. pMSCs according to the present invention can also be used for direct implantation by injecting the cells to the injury site. pMSCs canalso be stimulated by TNF-α or IFN-γ and produce extracellular vesicles, including exosomes, that can then be applied to the patients at certain dosage for immunomodulation actions. Finally, the soluble factors of the pMSCs contain proteins, such as basic fibroblast growth factors (bFGF), β-galactoside-specific lectins and interleukin 1B, which act as regulators of the immune response, cell growth, differentiation and angiogenesis.

What is claimed is:

1. A method of forming non-naturally occurring cells that exhibit the characteristics of naturally occuring perinatal mesenchymal stem cells, comprising the steps of:
culturing an amount of human induced pluripotent stem cells in a first medium for a first predetermined period of time to form an amount of human induced pluripotent stem trophoblasts;
differentiating the amount of human induced pluripotent stem trophoblasts in a second medium for a second predetermined period of time, wherein the second medium comprises an amount of an ALK5 inhibitor, an amount of BMP-4, and an amount of an FGFR3 inhibitor; and
replating the human induced pluripotent stem trophoblasts after the second predetermined period of time;
differentiating the replated human induced pluripotent stem trophoblasts in a third medium for a third predetermined period of time; and
replating the differentiated replated human induced pluripotent stem trophoblasts and further differentiating the replated human induced pluripotent stem trophoblasts for a fourth predetermined period of time to form an amount of non-naturally occurring cells that exhibit the characteristics of naturally occurring perinatal mesenchymal stem cells.

2. The method of claim 1, where the first medium comprises a stem cell culture medium.

3. The method of claim 2, where the first medium comprises a rock inhibitor.

4. The method of claim 3, wherein the first predetermined period of time comprises two days.

5. The method of claim 1, wherein the amount of the ALK5 inhibitor, the amount of BMP-4, and the amount of the FGFR3 inhibitor are present in a ratio of 10 μM of the ALK5 inhibitor to 10 ng/ml of BMP-4 to 0.1 μM of the FGFR3 inhibitor.

6. The method of claim 2, wherein the second predetermined period of time comprises four days.

7. The method of claim 1, wherein the third medium comprises a serum-free medium.

8. The method of claim 7, wherein the serum free medium encourages growth and expansion of human mesenchymal stem cells.

9. The method of claim 1, wherein the third medium includes a rock inhibitor.

10. The method of claim 9, wherein the third predetermined period of time comprises six days.

11. The method of claim 1, wherein the step of replating the differentiated replated human induced pluripotent stem trophoblasts and further differentiating the replated human induced pluripotent stem trophoblasts is repeated at least once.

12. The method of claim 1, wherein the step of replating the differentiated replated human induced pluripotent stem trophoblasts is performed at a cell seeding density of 1:5.

13. An amount of perinatal mesenchymal stem-like cells formed according to the method of claim 1.
